(19) United States
(12) Patent Application Publication
(10) Pub. No.: US 2026/0306156 A1
(43) Pub. Date: Oct. 8, 2026

(54) ORTHOPEDIC IMPACTING DEVICE

(71) Applicant: MEDACTA INTERNATIONAL SA, CASTEL SAN PIETRO (CH)

(72) Inventors: Francesco SICCARDI, CASTEL SAN PIETRO (CH); Massimiliano BERNARDONI, CASTEL SAN PIETRO (CH); Christian TROMBETTA, CASTEL SAN PIETRO (CH); Roberto NOSEDA, CASTEL SAN PIETRO (CH)

(21) Appl. No.: 19/481,068

(22) PCT Filed: May 29, 2024
(86) PCT No.: PCT/IB2024/055216

§ 371 Date: Nov. 3, 2025

(30) Foreign Application Priority Data
Jun. 1, 2023 (IT) 102023000011178

(51) Int. Cl. A61B 17/92

(52) U.S. Cl. CPC A61B 17/92; A61B 2017/925; A61B 2017/928

(57) ABSTRACT

An orthopedic impacting device comprises an electric motor, an actuating device, an anvil removably associated with a surgical tool to be introduced or extracted, a striker element adapted to interact with the anvil to promote the introduction or extraction of the surgical tool, and a motion transmitting system adapted to transfer the motion from the electric motor to the striker element. The anvil has two abutment surfaces interacting with the striker element, and said two abutment surfaces comprise a first pushing surface and a second extraction surface. The motion transmitting system comprises a pusher element, adapted to move the striker element by two elastic elements capable of amplifying the thrust exerted by the striker element selectively on one of the two abutment surfaces of the anvil. The anvil is an internally hollow cylinder within which the striker element and, within the latter, the pusher element are contained.

### SCOPE OF APPLICATION

The present invention relates to an orthopedic impacting device. In particular, the present invention is mainly used as an aid for the surgeon during the operation of introduction or extraction of prostheses or surgical tools, such as a broach inside the femoral canal.

Such a device, generically portable and battery-powered, is intended primarily to facilitate the surgeon in such manoeuvres, replacing the classic hand hammer.

### PRIOR ART

Several orthopedic impacting devices are known on the market.

Document EP3162314 describes a battery-powered electrical apparatus capable of imparting a direct thrust in two opposite directions, one used to push and the other to extract a surgical tool.

Functionally, the system consists of a piston moved by a connecting rod and crank; the piston acts on a chamber in which air is alternately compressed or depressurised; the striker element, hence the part that actually exerts the mechanical action on the anvil (or adapter to which the broach or other tool is mounted), is essentially a second, free piston that is moved by the compressed air generated by the first piston: the mass of the striker element and the speed it acquires results in the energy pulse that is transmitted.

The patent reports some functional variants, for example the use of valves to control the pressure generated and vary the size of the blow accordingly, or a catch to hold the striker element and release it only when the pressure reaches high values.

Instead, in order to obtain the blows in the opposite direction, used to extract the tool or otherwise, the patent describes a lever gear actuated by movable pins that are struck by the striker element. The striker element then hits a system of levers which, in turn, strike the head to exert the pressure, in the opposite direction, necessary to promote the extraction of the prosthesis or of the tool.

Such a system, while working well, can nevertheless be delicate due to the presence of the levers that can break under the high magnitude of the blows undergone.

In general, the currently known systems are cumbersome, constructively complicated and heavy, making them unwieldy for the surgeon. Furthermore, the impact force is not always correctly channeled and concentrated for its impulsive effect to be maximised, but is partly dispersed due to the presence of constructive actuations and motion transmissions. The known devices have numerous structural parts for motion actuation, which therefore, in addition to dispersing the imparted energy, are structurally delicate and, over time, subject to breakage.

In addition, in the systems with several mechanical actuation parts, precisely because of the increased fragility it is necessary to apply force less than the one it would actually be required, resulting in a less effective blow: consequently, it is necessary to increase the number of blows required to achieve the introduction or extraction of the tool or prosthesis.

An object of the present invention is to overcome the drawbacks of the prior art.

In particular, the object of the present invention is to propose an orthopedic impacting device that is constructively simple and easy to handle, in order to facilitate the surgeon's grip and use.

Therefore, it is the object of the present invention to provide an orthopedic impacting device that internally presents direct transmissions of the motion, without linkages or articulated actuations, so as to concentrate the force imparted to introduce or extract the surgical tool.

A further object of the present invention is to realise an orthopedic impacting device that is capable of imparting a direct blow at high energy in order to be able to reduce the number of blows necessary to complete the operation.

It is also the object of the present invention to provide an orthopedic impacting device that reduces surgical times and that is durable.

These and further objects and advantages are achieved by an orthopedic impacting device as illustrated in the attached claims.

### SUMMARY

A first aspect of the present invention provides for an orthopedic impacting device comprising an electric motor, an actuating device, an anvil that can be removably associated with a surgical tool to be introduced or extracted, a striker element adapted to interact with the anvil to promote the introduction or extraction of the surgical tool, and a motion transmitting system adapted to transfer the motion from the electric motor to the striker element. Advantageously, the anvil has two abutment surfaces for interaction with the striker element, which comprise a first pushing surface and a second extraction surface. The motion transmitting system comprises a pusher element, adapted to move the striker element by means of two elastic elements capable of amplifying the thrust exerted by the striker element selectively on one of the two abutment surfaces of the anvil.

Preferably, the anvil is an internally hollow cylinder within which the striker element and the pusher element are contained. Advantageously, the pusher element is in turn contained within the striker element.

The orthopedic impacting device comprises a containment sleeve within which the anvil, the striker element and the pusher element are housed.

Preferably, the pusher element, the striker element and the anvil are linearly movable with oscillatory motion along a longitudinal axis of the orthopedic impacting device.

The containment sleeve has a first open end, through which the anvil slides and exits at least partially, and a second end connected to a compartment within which the electric motor and, at least partially, the motion transmitting system are contained.

The anvil is capable of oscillating along the longitudinal axis within the impacting device between a retracted pushing position, wherein it is completely contained within the containment sleeve, and an extracted pulling position, wherein it is at least partially external to the containment sleeve.

The first pushing surface and the second extraction surface are parallel to each other, define the bases of the cylinder constituting the anvil and are orthogonal to the longitudinal sliding axis. The first pushing surface is located near the first end of the containment sleeve.

The motion transmitting system comprises a worm screw cooperating with a nut-screw operatively associated with the pusher element, for promoting axial oscillation, in both travel directions, of the pusher element, between a top dead centre located near the first end of the containment sleeve and a bottom dead centre located near the second end of the containment sleeve.

The striker element is moved by the pusher element to oscillate along with it, inside the anvil.

The pusher element, internally housed within the striker element and sliding axially therein, has a head portion between two elastic elements.

These elastic elements are a pair of coil springs, between which a head of the pusher element is interposed; the axial displacement of the pusher element, in cooperation with the coil springs, produces the oscillation along the axis of the striker element.

The pusher element is connected to the striker element only through the springs.

The motion transmitting system transforms the rotary motion of the electric motor into linear oscillatory motion of the pusher element, which in turn drags the striker element, by means of the coil springs, in the translation along the longitudinal axis. The elastic expansion action of the coil springs is capable of enhancing, like a sling effect, the impact force of the striker element against one of the two abutment surfaces of the anvil.

Advantageously, the direction of this linear oscillatory motion is determined by the direction of rotation of the worm screw.

Preferably, the striker element has a cylindrical shape with a first wall and a second wall opposite to the first and parallel to each other, defining the bases of the striker element. The first wall of the striker element impacts against the first pushing surface of the anvil, when the anvil is in the retracted position within the containment sleeve and the pusher element is at the top dead centre, to push the anvil outwardly of the impacting device.

The second wall of the striker element impacts against the second extraction surface of the anvil, when the anvil is in a position at least partially extracted from the containment sleeve and the pusher element is at the bottom dead centre, to pull the anvil and have it retract within the impacting device.

The pusher element performs an oscillation along the longitudinal axis with a constant excursion amplitude within the containment sleeve. The interaction and impact of the striker element against the first or second abutment surface of the anvil depends on the position of the anvil relative to the containment sleeve.

The orthopedic impacting device also comprises a pair of springs, placed around the anvil, between an outer surface of the anvil and an inner surface of the containment sleeve; the outer springs are adapted to maintain the anvil in a neutral position, intermediate between the retracted pushing position and the extracted extraction position.

Advantageously, the first wall of the striker element has a circular impact surface, while the second wall of the striker element has an annular impact surface.

### BRIEF DESCRIPTION OF THE DRAWINGS

The present invention will be become more evident from the following detailed description, with reference to the accompanying drawings provided by way of example only, wherein:

FIG. 1 illustrates a perspective view of an impacting device in accordance with the present invention;

FIG. 2 illustrates a perspective view sectioned along a longitudinal plane of the orthopedic impacting device in FIG. 1;

FIG. 3 illustrates a lateral section along a longitudinal plane of the orthopedic impacting device subject-matter of the present invention in a non-operational neutral configuration;

FIG. 4 illustrates an enlargement of a first portion of the sectioned view illustrated in FIG. 3;

FIG. 5 illustrates an enlargement of a second portion of the sectioned view illustrated in FIG. 3;

FIGS. 6 and 7 illustrate a lateral section along a longitudinal plane of the orthopedic impacting device subject-matter of the present invention in two different operational steps: FIG. 6 of extraction and FIG. 7 of introduction.

### DETAILED DESCRIPTION

In the above-mentioned figures, an orthopedic impacting device in accordance with the present invention has been indicated as a whole with 1.

The orthopedic impacting device 1 comprises a containment casing or case 2, preferably developing along a longitudinal axis 2a, within which an energy source 3, such as an electric motor, is contained.

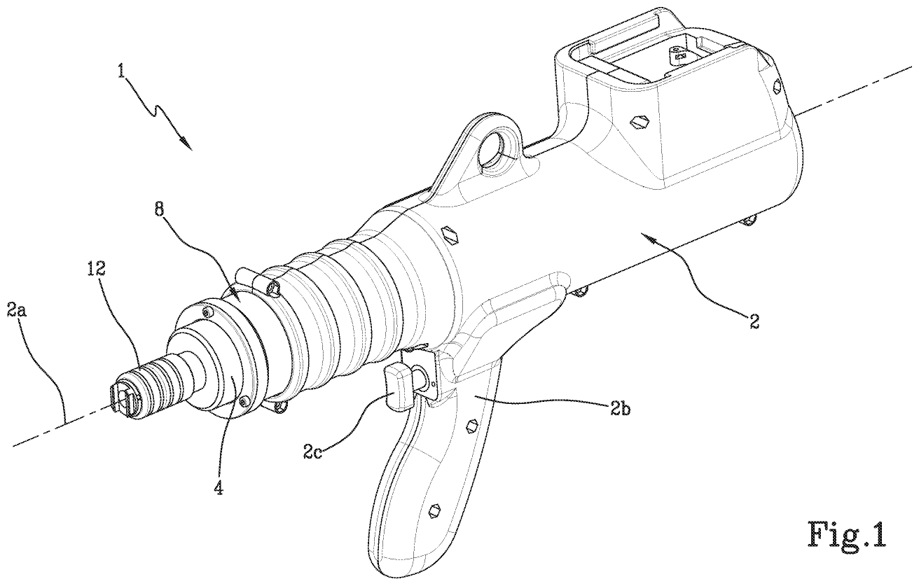

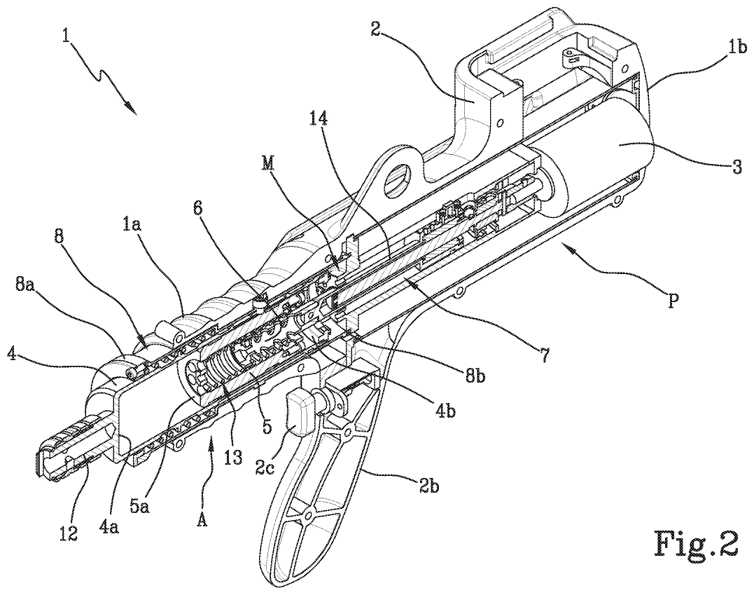

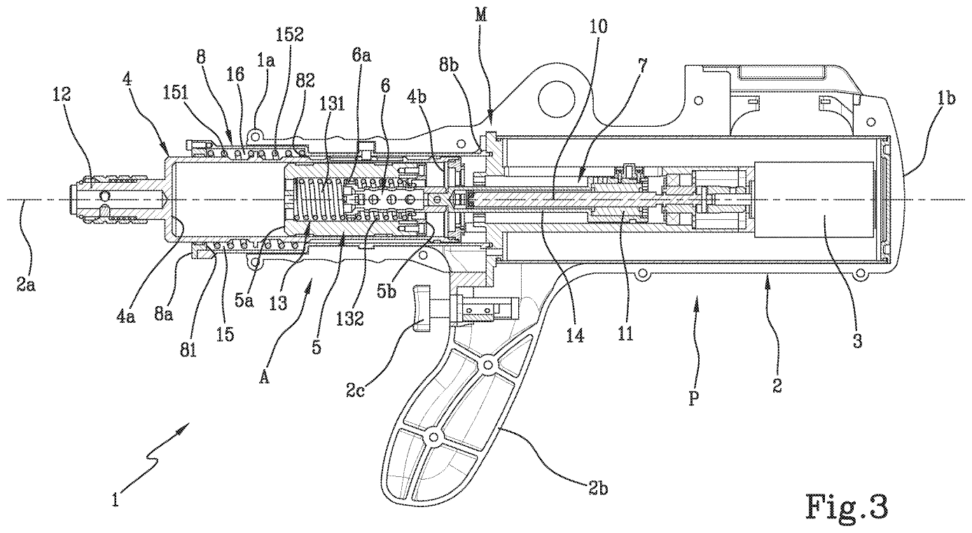

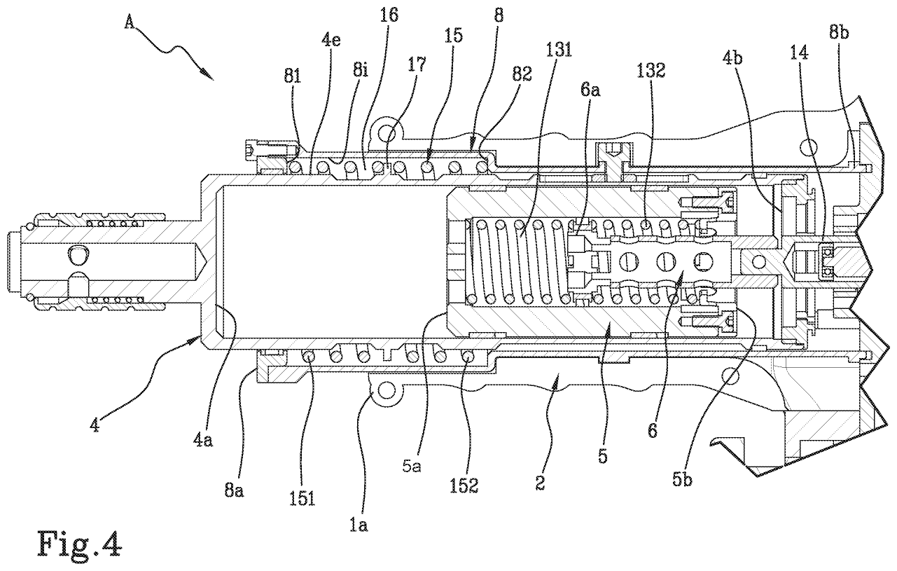

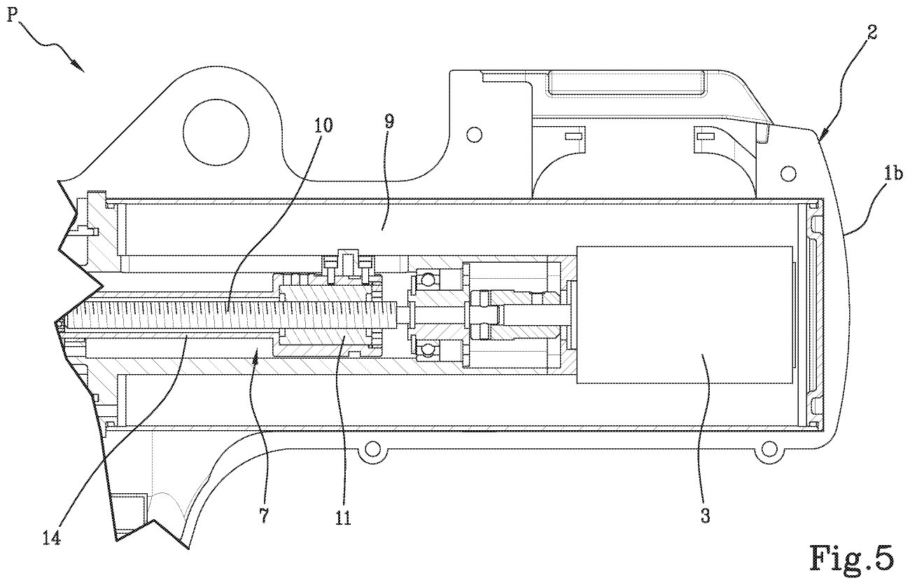

In a preferred embodiment illustrated in the annexed figures, with particular reference to FIGS. 1 and 2, the orthopedic impacting device 1 has an electric drill shape with a main central body extending along the longitudinal axis 2a and a handle 2b, provided with an actuating device 2c. As is visible in FIGS. 3, 6 and 7, internally the orthopedic impacting device 1 can be subdivided into two portions: a rear portion P (illustrated enlarged in FIG. 5), within which the electric motor 3 is housed, and a front portion A (illustrated enlarged in FIG. 4), within which the movable and oscillating parts adapted to create the actual striking and impact effect are housed.

As is best visible in FIG. 4, in the front portion A there is a containment sleeve 8 while the rear portion P, which is best visible in FIG. 5, contains a housing compartment 9 for the electric motor 3 and for a motion transmitting system 7. The containment sleeve 8 and the housing compartment 9 are connected to each other internally to the containment casing 2 of the orthopedic impacting device, essentially in an intermediate zone M along the longitudinal development of the orthopedic impacting device 1.

In the front portion A, the orthopedic impacting device 1 comprises an anvil 4 reversibly associated with a surgical tool, such as a broach, which must be introduced into or extracted from the human body. The anvil 4 is movable along the longitudinal axis 2a between two extreme positions: a first position at which it is completely contained in the containment casing 2, and a second position at which it is at least partially extracted from the containment casing 2. The anvil 4 exits from the containment casing 2 through a first end 8a of the containment sleeve 8.

Advantageously, the anvil 4 is an internally hollow cylinder as will be described later.

The orthopedic impacting device 1 has an open first end 1a, through which the containment sleeve 8 and the anvil 4 partially protrude. This first end 1a is thus facing the surgical tool to be introduced or extracted. The orthopedic impacting device 1 also comprises a second end 1b, opposite to the first and delimiting the rear zone P.

The orthopedic impacting device 1 further comprises a striker element 5 adapted to interact with the anvil 4 for promoting its thrust and traction in the ways that will be described below and thus, in general, for introducing or extracting the surgical tool.

As shown in the attached figures, the striker element 5 is internally housed within the containment casing 2. More in detail, the striker element 5 is housed within the anvil 4.

The orthopedic impacting device also comprises a motion transmitting system 7 adapted to transfer the motion from the electric motor 3 to the striker element 5. Specifically, the motion transmitting system 7 comprises a pusher element 6 which is capable of actuating the striker element 5.

The striker element 5 is then set in motion, still with an oscillatory movement along the axis 2a, by said pusher element 6. The movement of the striker element 5 and of the pusher element 6 are both of the translational type along the longitudinal axis 2a, for promoting the thrust of the anvil 4 along said longitudinal axis 2a. Specifically, the striker element 5 and the pusher element 6 both move linearly with oscillatory motion along the longitudinal axis 2a. This movement of the striker element 5 against the anvil 4 results in a linear oscillatory movement along the longitudinal axis 2a of the anvil 4 itself.

The electric motor 3 actuates the motion transmitting system 7, which is capable of transferring the motion from the electric motor 3 to the pusher element 6 and, thus, of activating the striker element 5.

The motor 3 is advantageously managed by electronic boards and is activated by pressing a button or actuating device 2c located in the handle of the impacting device itself. The energy delivered is adjusted by modulating the stroke of the button 2c.

The possibility of operation is either by single pulse, or by continuous action by acting on the button pressure time.

As has been described above, the anvil 4, the striker element 5 and the pusher element 6 are housed within the containment casing 2 of the impacting device. More specifically, within the containment casing 2 of the impacting device, positioned in the front portion A, there is the aforementioned containment sleeve 8 within which the anvil 4, the striker element 5 and the pusher element 6 are housed.

The containment sleeve 8 has a first open end 8a, through which the anvil 4 slides and exits at least partially, and a second, at least partially open end 8b, opposite to the first one.

The front portion A of the orthopedic impacting device 1 thus develops over the longitudinal length occupied by the containment sleeve 8, while the rear portion P coincides with the housing compartment 9 of the electric motor and of the motion transmitting system 7.

In detail and with reference to FIGS. 4 and 5, the motion transmitting system 7 comprises a worm screw 10 cooperating with a nut-screw 11, operatively associated with the pusher element 6. The electric motor 3 actuates the worm screw 10 which, by rotating on itself around the longitudinal axis 2a, causes the translation of the nut-screw 11 which, in turn, is trapped inside a transmission sheath 14. The transmission sheath 14 is, in turn, connected to the pusher element 6 causing its translation along the longitudinal axis 2a, in one or the other direction depending on the direction of rotation of the worm screw 10.

In this way, the pusher element 6 can oscillate axially, in both travel directions along the longitudinal axis 2a, between a top dead centre TDC, turned towards the first end 1a of the orthopedic impacting device 1 (or the first end 8a of the containment sleeve 8) and a bottom dead centre BDC turned towards the second end 8b of the containment sleeve 8.

The anvil 4 comprises two abutment surfaces 4a and 4b, each adapted to interact with a respective wall of the striker element 5; these two abutment surfaces 4a and 4b comprise, respectively, a first pushing surface 4a, located anteriorly and close to the first open end 8a of the containment sleeve 8 and, therefore, of the first end 1a of the orthopedic impacting device 1, and a second extraction surface 4b. The first pushing surface 4a passes through the open end of the sleeve and of the orthopedic impacting device. The extraction surface 4b, on the other hand, is located near the intermediate zone M, close to the second end 8b of the containment sleeve 8. The two abutment surfaces 4a and 4b are parallel to each other, located at opposite ends of the anvil 4 and are orthogonal to the longitudinal axis 2a of translation of the motion passing through their central point. The anvil 4 preferably has a cylindrical shape, and the aforementioned abutment surfaces define the end walls of this cylinder. Advantageously, the pushing surface 4a has a solid circular shape and externally has a pin 12 projecting axially away from the anvil. This pin 12 allows connection with a surgical tool or a prosthesis.

The extraction surface 4b has instead a substantially annular shape since, centrally, there must be room for the passage of the motion transmitting system 7, in particular for the screw 10 and the sheath 14.

The striker element 5, in turn, comprises a first wall 5a or front wall and a second wall 5b, opposite to the first, or rear wall. Advantageously, the striker element 5 is internally hollow.

The striker element 5 preferably has a cylindrical shape.

The walls of the striker element 5 also have a circular shape in the front part, so the first wall 5a turned towards the first end 1a of the impacting device and interacting with the pushing surface 4a of the anvil is circular, while the rear or second wall 5b has an annular shape, leaving the central zone open for the passage of the motion transmitting system 7. The first wall or front wall 5a of the striker element 5 interacts and impacts against the pushing surface 4a of the anvil 4, while the second wall or rear wall 5b of the striker element 5 interacts and impacts against the extraction wall 4b of the anvil 4.

The striker element 5 is contained within the anvil 4 just as the pusher element 6 is floating and contained within the striker element 5.

In other words, the anvil 4 is an internally hollow cylinder within which the striker element 5 and, within the striker element 5, the pusher element 6 are contained, nested within each other. Anvil 4, striker element 5 and pusher element 6 are coaxial, concentric and inserted into each other.

The pusher element 6 is floating within the striker element 5, between two elastic elements 13 which amplify the thrust of the striker element 5 on the anvil 4, as will be described below.

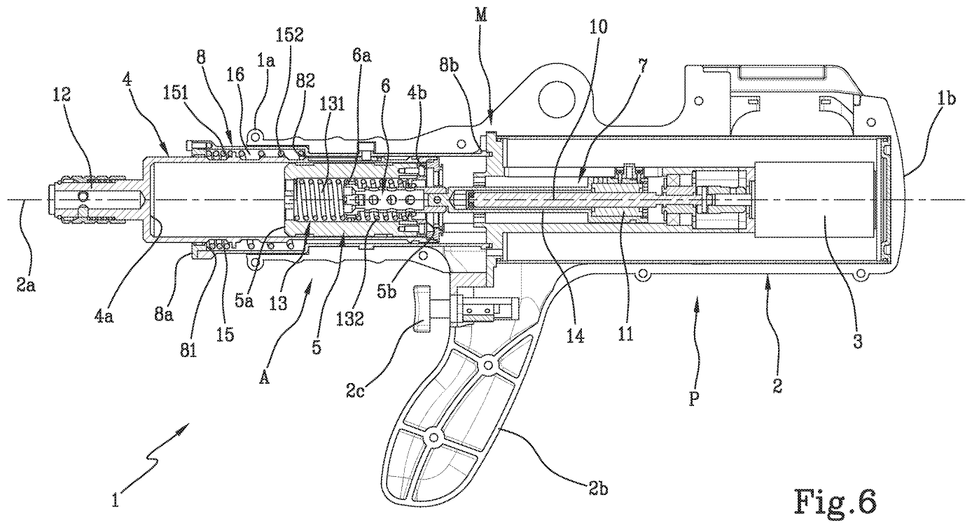

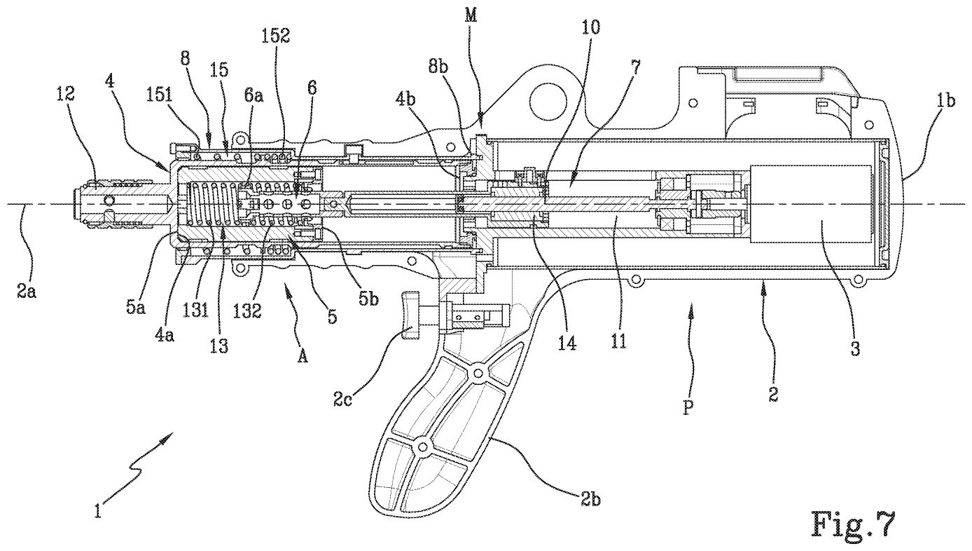

The anvil 4 is capable of oscillating and translating along the longitudinal axis 2a within the containment casing or case 2 of the impacting device; specifically, the anvil 4 is capable of oscillating and translating along the longitudinal axis 2a within the containment sleeve 8, between a completely retracted pushing position (FIG. 7), wherein it is completely contained within the orthopedic impacting device 1, and an extracted pulling position (FIG. 6), wherein it is at least partially external to the orthopedic impacting device 1.

As shown in FIG. 7, the anvil 4 is in a completely retracted position (hence completely introduced into the sleeve 8) as the surgeon pushes the impacting device 1 against the patient, thus against the surgical tool to be introduced: the first wall 5a of the striker element 5 impacts against the pushing surface 4a of the anvil 4, both of which are located near the first end 1a of the orthopedic impacting device 1, acting as a sort of hammer against the anvil 4, which is therefore pushed outwardly of the containment sleeve 8, thereby pushing the surgical tool through the connection with the connecting pin 12; in this way, the introduction of the tool itself into place within the human body is favoured.

On the contrary, as shown in FIG. 6, if the surgical tool is to be extracted, the surgeon pulls the impacting device towards him-/herself, away from the patient, causing the at least partial extraction of the anvil 4 which is connected, through the pin 12, to the surgical tool in turn introduced into the human body. The relative movement in translation between the anvil and the sleeve of the impacting device causes, by pulling the impacting device away from the body, the anvil 4, which is connected to the tool to be extracted from the human body, to exit at least partially from the sleeve 8. In this extracted or at least partially extracted position of the anvil (FIG. 6), the second wall 5b of the striker element 5, which thus interacts with the extraction surface 4b of the anvil, comes into play. By impacting against the latter, the striker element 5 pulls the anvil 4 along with it towards the inside of the impacting device 1, which then promotes the extraction of the surgical tool connected to it by means of the pin 12. In other words, the pushing action of the striker element against the extraction surface 4b of the anvil is similar to that of a hammer, but which now acts in the opposite direction to the introduction direction described in the previous situation of FIG. 7, thus acting in extraction. The surgical tool is thus pulled by the pin 12, which is connected to anvil 4: the latter is in turn pulled away from the human body, thus causing the surgical tool to be extracted.

The motion transmitting system 7 comprises and is connected to the pusher element 6, which may be considered as floating in the sense that it is held raised by the worm screw 10, and in particular by the transmission sheath 14, without being directly connected to the striker element 5.

The striker element 5 is moved by the pusher element 6 to oscillate along with it, within the anvil 4.

In fact, the pusher element 6 is connected to the striker element 5 by means of two elastic elements 13, which are capable of amplifying the thrust of the pusher element 6 exerted on the striker element 5 and thus capable of amplifying the impact force of the striker element 5 selectively on one of the two abutment surfaces 4a and 4b of the anvil 4, depending on whether the tool is to be introduced or extracted.

In particular, the pusher element 6, internally housed within the striker element 5 and sliding axially therein, comprises a head 6a between the aforementioned elastic elements 13.

In other words, two elastic elements 13 and the pusher element 6 are housed within the striker element 5.

These elastic elements 13 are, preferably, a pair of coil springs, between which the aforementioned head 6a of the pusher element 6 is interposed.

A first spring 131 is placed between the first end 5a of the striker element 5 and the head 6a of the pusher element 6, the second spring 132 is placed between the head 6a and the second end 5b of the striker element, both placed within the striker element 5.

The axial displacement of the pusher element 6, in cooperation with the coil springs 131 and 132, produces the oscillation of the striker element 5 along the longitudinal axis 2a. The pusher element 6 is connected to the striker element 5 only through the two coil springs 131 and 132.

Thus, the rotation of the electric motor 3 and of the worm screw 10 are transformed, through the nut-screw 11, into translatory motion to the pusher element though the transmission sheath 14 and, thus to the striker element 5. The elastic expansion action of the coil springs 131 and 132, then, is capable of enhancing, like a sling effect, the impact force of the striker element 5 against one of the two abutment surfaces 4a or 4b of the anvil 4.

The translation of the striker element shows an oscillatory motion in both directions along the longitudinal axis 2a as a result of the reversal of the direction of rotation of the worm screw 10.

The oscillation of the pusher element 6 within the containment sleeve 8 has a constant excursion amplitude: the interaction and impact of the striker element 5 against the first 4a or the second 4b abutment surface of the anvil 4 depends on the position of the anvil 4 relative to the containment sleeve 8.

The impacting device 1 further comprises further elastic elements 15 placed around the anvil 4, within the gap 16 placed between the anvil 4 and the containment sleeve 8, in particular between the outer surface 4e of the anvil 4 and the inner surface 8i of the containment sleeve 8.

These further elastic elements 15 preferably comprise a pair of coil springs 151 and 152 separated by an annular septum 17 protruding radially from the outer surface of the anvil 4.

The first spring 151 is between a front shoulder 81 of the sleeve 8 and the septum 17 and the second spring 152 is between said septum 17 and a rear shoulder 82 of the sleeve 8.

These coil springs 151 and 152, placed around the anvil 4, serve to keep the anvil 4 in a neutral position (illustrated in FIG. 3), in which there is no interaction between striker element 5 and anvil 4.

Advantageously, there is also provided a sensor, not depicted in the drawings, which is capable of detecting the position of the anvil and of activating the motor to turn the screw in one or the other direction in order to actuate the striker element in one or the other direction.

The following situations emerge during use.

Situation of introduction of the surgical tool within the human body. Starting from the neutral position of the anvil (visible in FIG. 3), in which the outer coil springs 15, those placed around the anvil 4, keep the anvil in an intermediate position between the retracted introduction position and the extracted extraction position, the surgeon pushes on the impacting device by pressing the broach engaged on the pin 12 against the bone. The anvil then retracts passing from the intermediate position to a completely retracted position introduced within the sleeve 8 (FIG. 7). In fact, the second coil spring 152, under the pressure exerted by the surgeon on the impacting device, compresses, thus bringing the anvil into a retracted position so as to be able to actuate the thrust or abutment of the striker element on the pushing surface of the anvil. In fact, in this completely retracted position within the sleeve, the sensor detects the position of the anvil and sends a signal to the motor, which is actuated to activate the motion transmitting system, which then actuates the striker element, unlocking the movement of the entire device. Since the anvil is in a completely retracted position introduced into the containment sleeve, the striker element interacts with the anvil by bringing its first surface into abutment against the first surface of the anvil, both of which placed towards the first open end of the impacting device (FIG. 7).

The tool is then introduced.

When it is wished to interrupt the thrust, the surgeon stops pressing against the bone and the anvil returns to a neutral position: the sensor detects the new position of the anvil and the striking mechanism stops.

When it is wished to extract the tool, the entire impacting device must be pulled or made to retract. The surgeon pulls the device towards him/her to extract the broach, this causes the second coil spring 152 to stretch and, since the broach is wedged in the bone, the anvil is also dragged outwards (FIG. 6) because it tends to remain stuck to the bone.

The sensor thus frees the movement of the striking mechanism given the advancement position outwardly of the anvil, and it is proceeded with the extraction. As the anvil is in an at least partially extracted position, the striker element will abut with its second abutment surface, against the second abutment surface of the anvil, both surfaces placed towards the intermediate zone of the orthopedic impacting device (FIG. 6).

The invention achieves its intended purposes because it offers an extremely handy, lightweight, constructively simple and robust orthopedic impacting device thanks to its internal motion actuation structure free of linkages that make the structure delicate.

The impacting device subject-matter of the present invention makes it possible to apply a considerable force of impact, which is constant and in a concentrated manner without energy dissipation, thanks to the presence of an elastic support, such as two coil springs, which increases and enhances the force of impact of the striker element selectively against the pushing or extraction surface as it dynamically amplifies the speed of the striker element during the impacting step. The speed of the pusher element follows an established and optimised profile to maximise impact performance.

In this way, the number of blows to be imparted in order to complete the required operation can also be reduced.

The orthopedic impacting device described makes the surgical procedure simpler and easier for the surgeon by replacing the traditionally used hammer with handle.

The impacting device in question, equipped with a battery-powered electric motor, releases energy causing the impacting device to apply a selectively controlled force on the two impact surfaces of the anvil to create a precise impact at its end. This impact operates alternately in two opposite directions depending on the relative position between the anvil and the impacting device, thus generating an introduction or extraction force.

1. An orthopedic impacting device comprising an electric motor, an actuating device, an anvil that can be removably associated to a surgery tool to be introduced or extracted, a striker element adapted to interact with said anvil to promote introduction or extraction of the surgery tool, a motion transmitting system adapted to transfer the motion from the electric motor to the striker element; wherein:
said anvil has two abutment surfaces interacting with said striker element, said two abutment surfaces comprising one first pushing surface and one second extraction surface,
said motion transmitting system comprises a pusher element, adapted to move said striker element by means of two elastic elements capable of amplifying thrust exerted by the striker element selectively on one of said two abutment surfaces of said anvil, and
said anvil is an internally hollow cylinder within which the striker element and, inside the striker element, the pusher element are contained.

2. The orthopedic impacting device according to claim 1, wherein the orthopedic impacting device comprises a containment sleeve within which said anvil, said striker element, and said pusher element are housed.

3. The orthopedic impacting device according to claim 1, wherein said pusher element, said striker element, and said anvil are linearly movable with oscillatory motion along a longitudinal axis of said orthopedic impacting device.

4. The orthopedic impacting device according to claim 2, wherein said containment sleeve has a first open end, through which said anvil slides and exits at least partially, and a second end connected to a compartment within which said electric motor and, at least partially, said motion transmitting system are contained.

5. The orthopedic impacting device according to claim 2, wherein said anvil is capable of oscillating along a longitudinal axis of said orthopedic impacting device within said orthopedic impacting device between a retracted pushing position, wherein the anvil is completely contained within said containment sleeve, and an extracted pulling position, wherein the anvil is at least partially external to said containment sleeve.

6. The orthopedic impacting device according to claim 5, wherein said first pushing surface and said second extraction surface are parallel to each other, define bases of the cylinder constituting said anvil and are orthogonal to said longitudinal axis of said orthopedic impacting device, said first pushing surface being placed near a first open first end of said containment sleeve.

7. The orthopedic impacting device according to claim 2, wherein said motion transmitting system comprises a worm screw cooperating with a nut-screw operatively associated with said pusher element, for promoting axial oscillation, in both travel directions, of said pusher element, between a top dead centre (TDC) located near a first open end of said containment sleeve and a bottom dead centre (BDC) located near a second end of said containment sleeve.

8. The orthopedic impacting device according to claim 1, wherein said striker element is moved by said pusher element to oscillate along with said pusher element within said anvil.

9. The orthopedic impacting device according to claim 8, wherein said pusher element is internally housed within said striker element and axially slidable therein, said pusher element having a head portion between said two elastic elements.

10. The orthopedic impacting device according to claim 9, wherein said elastic elements are a pair of coil springs between which a head of said pusher element is interposed, and axial displacement of said pusher element, in cooperation with said coil springs, produces the oscillation along an axis of said striker element.

11. The orthopedic impacting device according to claim 10, wherein said pusher element is connected to said striker element only by said coil springs.

12. The orthopedic impacting device according to claim 1, wherein said motion transmitting system transforms the rotary motion of said electric motor into a linear oscillatory motion of said pusher element, which in turn drags said striker element, by means of a pair of coil springs, in translation along a longitudinal axis, wherein elastic expansion action of said coil springs is capable of enhancing, like a sling effect, an impact force of said striker element against one of the two abutment surfaces of said anvil.

13. The orthopedic impacting device according to claim 7, wherein the direction of linear oscillatory motion is determined by the direction of rotation of the worm screw.

14. The orthopedic impacting device according to claim 2, wherein said striker element has a cylindrical shape with a first wall and a second wall opposite to the first wall and parallel to each other, defining bases of said striker element; said first wall of said striker element impacting against said first pushing surface of said anvil, when said anvil is in a retracted position within said containment sleeve and said pusher element is in a top dead center (TDC) position, to push said anvil outwardly of said impacting device.

15. The orthopedic impacting device according to claim 14, wherein said second wall of said striker element impacts against said second extraction surface of said anvil, when said anvil is in a position at least partially extracted from said containment sleeve and said pusher element is in a bottom dead center (BDC), to pull said anvil and cause it to retract within said impacting device.

16. The orthopedic impacting device according to claim 1, wherein a pusher element performs an oscillation along a longitudinal axis of the orthopedic impacting device having a constant excursion amplitude within a containment sleeve, the interaction and impact of said striker element against said first or said second abutment surface of said anvil depends on a position of the anvil relative to said containment sleeve.

17. The orthopedic impacting device according to claim 2, wherein it comprises a pair of springs, placed around said anvil, between an outer surface of said anvil and an inner surface of said containment sleeve; said outer springs being adapted to maintain said anvil in a neutral position, intermediate between a retracted pushing position and a extracted extraction position.

18. The orthopedic impacting device according to claim 14, wherein said first wall of said striker element has a circular impact surface, while said second wall of said striker element has an annular impact surface.
